(19) United States
(12) Patent Application Publication
(10) Pub. No.: US 2023/0167483 A1
(43) Pub. Date: Jun. 1, 2023

(54) SYSTEM AND DEVICE FOR ANALYZING A SAMPLE

(71) Applicant: MIDGE MEDICAL GMBH, Berlin (DE)

(72) Inventors: Markus RIESTER, Wiesbaden (DE); Michael DIEBOLD, Berlin (DE)

(21) Appl. No.: 17/995,732

(22) PCT Filed: Apr. 7, 2021
(86) PCT No.: PCT/EP2021/059105

§ 371 Date: Oct. 7, 2022

(30) Foreign Application Priority Data
Apr. 7, 2020 (DE) 10 2020 109 744.1

(51) Int. Cl. C12Q 1/68; B01L 3/00; G01N 21/64

(52) U.S. Cl. CPC C12Q 1/68; B01L 3/502; G01N 21/64; B01L 2200/04; B01L 2200/10; B01L 2200/16; B01L 2200/025; B01L 2200/026; B01L 2300/042; B01L 2300/044; B01L 2300/087; B01L 2300/0663; B01L 2300/0672; B01L 2300/1805

(57) ABSTRACT

The invention relates to a system is provided that comprises a lysis chamber, an amplification chamber and a fluorescence detection device. The fluorescence detection device (16) comprises - a detection chamber (42) configured to receive the amplification chamber (14) or the contents of the amplification chamber, - a light source (44), - an optical sensor (46), - energy supply means (48), - a wireless data interface (52) and - a controller (50).

The invention relates to a system and a device for detecting a target analyte, in particular a target nucleic acid, for instance DNA or RNA, by way of isothermal nucleic acid amplification and fluorescence.

Nucleic acid amplification technologies are used to amplify the amount of a target nucleic acid in a sample in order detect such target nucleic acid in the sample. A known nucleic acid amplification technology is Polymerase Chain Reaction (PCR). Isothermal nucleic acid amplification technologies offer advantages over polymerase chain reaction (PCR) in that they do not require thermal cycling or sophisticated laboratory equipment.

Known isothermal nucleic acid amplification technologies are inter alia Recombinase Polymerase Amplification (RPA) and Strand Invasion Based Amplification (SIBA) and other methods know to persons skilled in the art.

Recombinase polymerase amplification (RPA), is a method to amplify the amount of a target analyte, in particular a nucleic acid such as DNA or RNA in a sample. For Recombinase polymerase amplification three core enzymes are used: a recombinase, a single-stranded DNA-binding protein (SSB) and a strand-displacing polymerase. Recombinases can pair oligonucleotide primers with homologous sequences in duplex DNA. SSB binds to displaced strands of DNA and prevents the primers from being displaced. The strand-displacing polymerase begins DNA synthesis at sites where the primer has bound to the target DNA. Thus, if a target gene sequence is indeed present in the tested sample, an exponential DNA amplification reaction can be achieved to amplify a small amount of a target nucleic acid to detectable levels within minutes at temperatures between 37° C. and 42° C.

The three core RPA enzymes can be supplemented by further enzymes to provide extra functionality. Addition of exonuclease III allows the use of an exo probe for real-time, fluorescence detection. If a reverse transcriptase that works at 37 to 42° C. is added then RNA can be reverse transcribed and the cDNA produced amplified all in one step.

By adding a reverse transcriptase enzyme to an RPA reaction, it can detect RNA as well as DNA, without the need for a separate step to produce cDNA. It is an advantage of RPA that it is isothermal and thus only requires simple equipment. While RPA operates best at temperatures of 37-42° C. it still works at room temperature.

For detecting the presence of a targeted nucleic acid in a sample, fluorescence detection technique can be used. After the light source at specific wavelength illuminates on the targeted nucleic acids, the DNA-binding dyes or fluorescein- binding probes of the nucleic acids will react and enable fluorescent signals to be emitted. The fluorescent signal is an indication of the existence of the targeted nucleic acids.

It is an object of the invention facilitate the testing of samples by means of nucleic acid amplification technology.

According to a first aspect of the invention, a system is provided that comprises a lysis chamber, an amplification chamber and a fluorescence detection device.

The lysis chamber may contain a lysing fluid that causes lysing of the cells in a sample to thus release the nucleic acids (DNA or RNA). The lysing fluid may comprise an acid, e.g. HCl or a weak alkali, and a surface active agent.

The amplification chamber contains a mixture that comprises a recombinase, a single-stranded DNA-binding protein (SSB) and strand-displacing polymerase that causes a recombinase polymerase amplification (RPA). The amplification chamber further contains exonuclease III allows the use of an exo probe for real-time, fluorescence detection.

Preferably, the system also comprises a piston with a lid for closing the lysis chamber and the amplification chamber.

The combination of the lysis chamber and piston is arranged such that in the event of the piston moving into the lysis chamber pressure release is possible through venting. The venting means are configured to prevent content of either the lysis chamber or the amplification chamber or both from escaping out of the respective chamber or chambers.

The system is configured to implement an isothermal amplification method such as RPA and SIBA and other methods, preferably isothermal methods. The amplification method is configured to be carried out in a temperature range between 25° C. and 47° C. In various embodiments, initial heating may be applied. Other embodiments implement continuous heating. There are also embodiments without any external heating. in one or more of the method steps.

The fluorescence detection device comprises
a detection chamber configured to receive the amplification chamber or the contents of the amplification chamber,
a light source,
an optical sensor,
an energy supply,
a wireless data interface and
a controller.

The controller can be a microcontroller and/or a state machine.

The fluorescence detection device may further comprise heating means that allow heating of an amplification chamber that is inserted in the fluorescence detection device.

The system can either be a point of care (POC) system, wherein the fluorescence detection device is arranged at a point of care, for instance in a medical doctor’s office. Alternatively, the system may be a personal system, wherein the fluorescence detection device is selfcontained and mobile, in particular pocketable.

A further aspect of the invention is a set of at least two distinct chambers that can be combined to form a single, fluid tight assembly, wherein a first chamber comprises a first set of chemicals and/or agents, said first chamber being closed prior to use, and wherein a second chamber comprises a second set of chemicals and/or agents that are at least in part distinct from the chemicals and/or agents of the first set, and wherein the first chamber comprises a lid, that can be opened when the first chamber and the second chamber are combined to form a single, fluid tight assembly, in order to allow the contents of the first chamber to enter the second chamber.

Preferably each chamber is an integral unibody. In an alternative embodiment, the assembly of the first chamber and the second chamber is an integral unibody.

Preferably, the chambers can be connected by means of a fluid tight snap fit connection to thus form a single, fluid tight assembly. As an alternative to a snap fit connection, a screw lock connection similar to a Luer-lock can be provided for tightly connecting the chambers. Another alternative is a press fit connection between the chambers.

The advantage of a fluid tight assembly of at least two chambers is that the assembly can be disposed easily without the risk of contamination because the contents of the assembly is tightly secured within the assembly.

Preferably, the second chamber has transparent walls that allow excitation and detection of luminescence and in particular fluorescence.

An assembly comprising two initially separate chambers and a fluorescence detection device with a receptacle that can receive the assembly for luminescence detection makes it possible to perform a testing method that comprises two consecutive chemical or biochemical method steps — for instance lysis and amplification — and a luminescence testing step in a simple, clean and safe manner that minimizes the risk of contamination and infection while providing easy handling. The fluorescence detection device can be re-used because in use it is not contacted by the sample or any agents or chemicals since these are tightly enclosed in the assembly of chambers. The assembly of chambers and its contents can be safely disposed after use because the contents is reliably kept within the interior of the assembly.

The invention shall now further be illustrated by way of an example and with a reference to the figures. Of the figures,

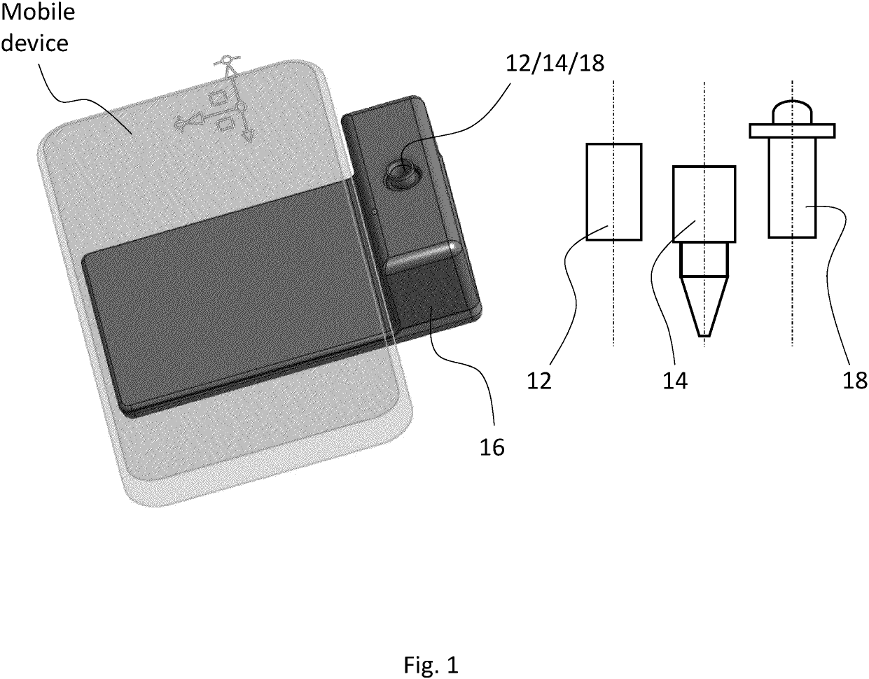

FIG. 1: shows a detection system according to the invention;

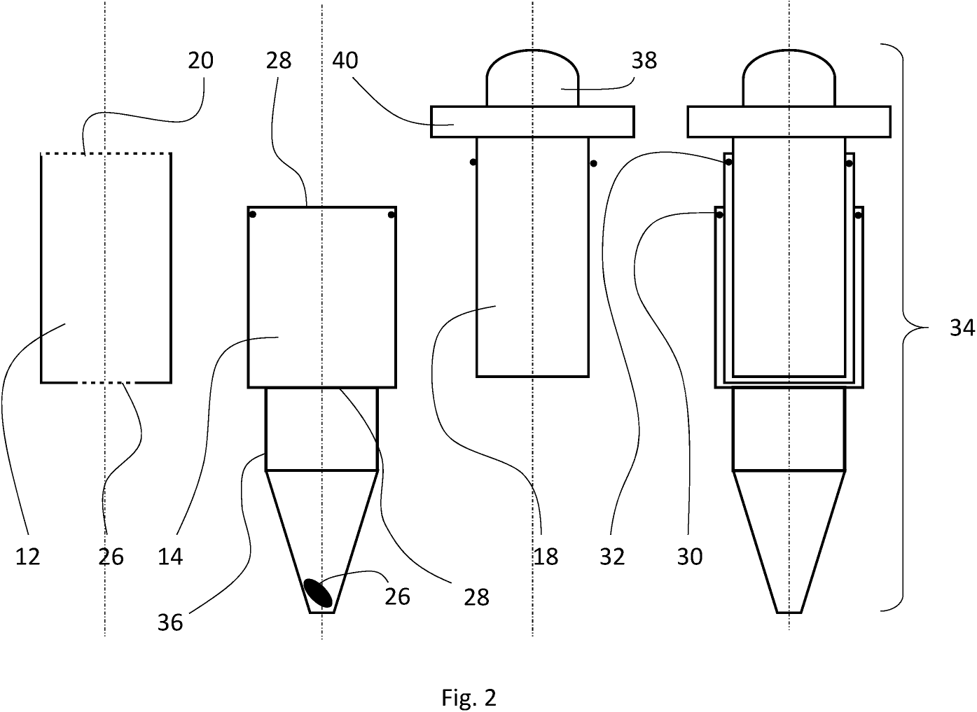

FIG. 2: shows a detection device of the detection system of FIG. 1;

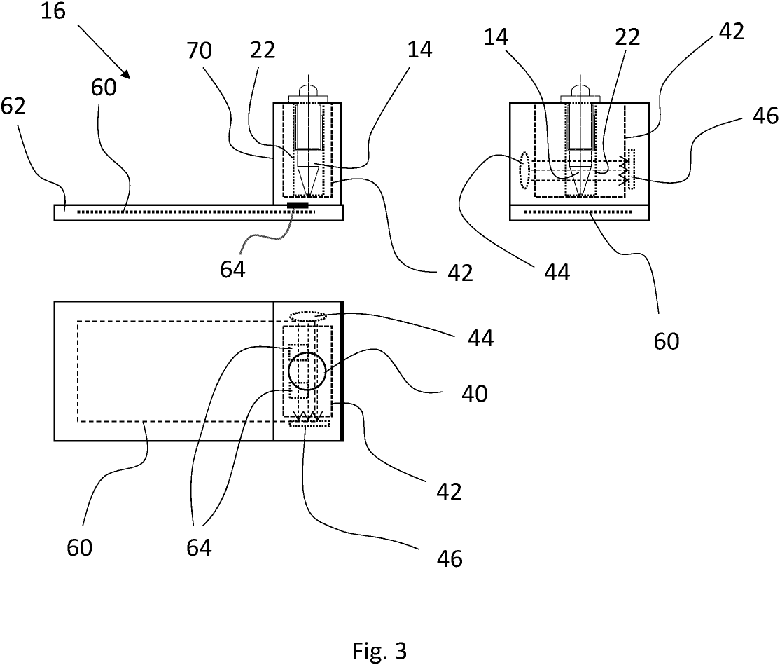

FIG. 3: shows a schematic diagram of the detection device of FIG. 2;

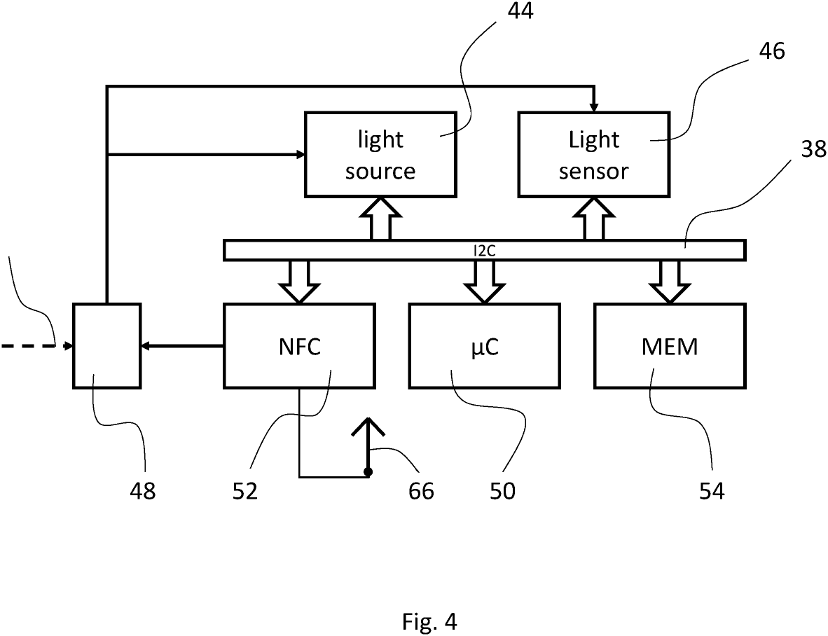

FIG. 4: is a cross-sectional view of the detection device of FIG. 2; and

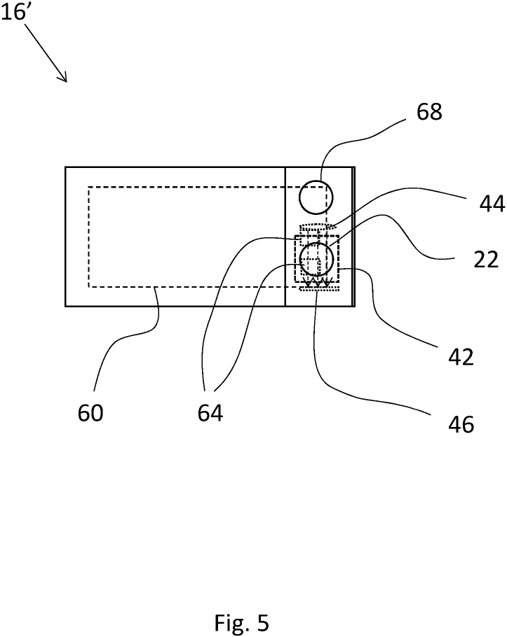

FIG. 5: is a schematic diagram of an alternative detection device

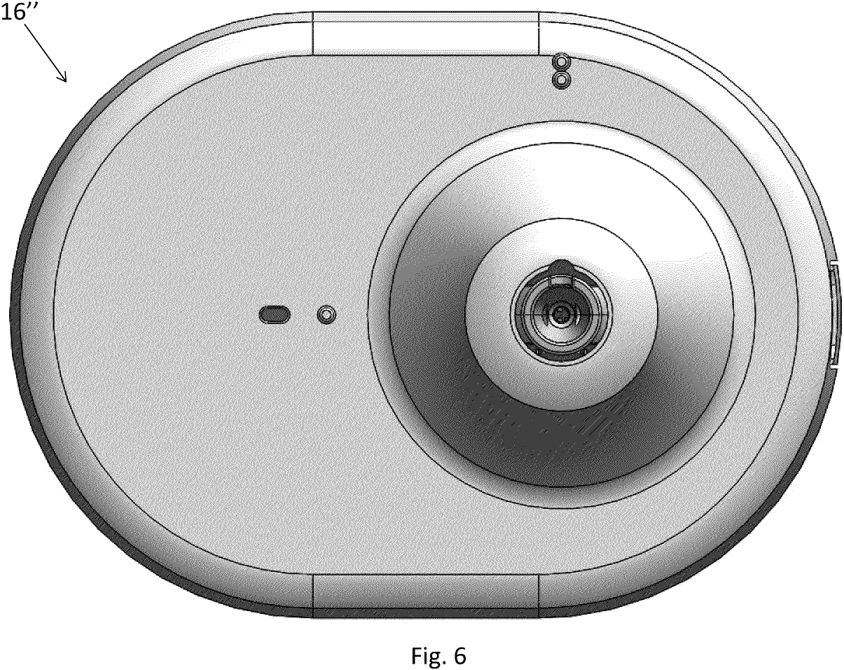

FIG. 6: is a top view of an alternative embodiment of the testing device according to the invention;

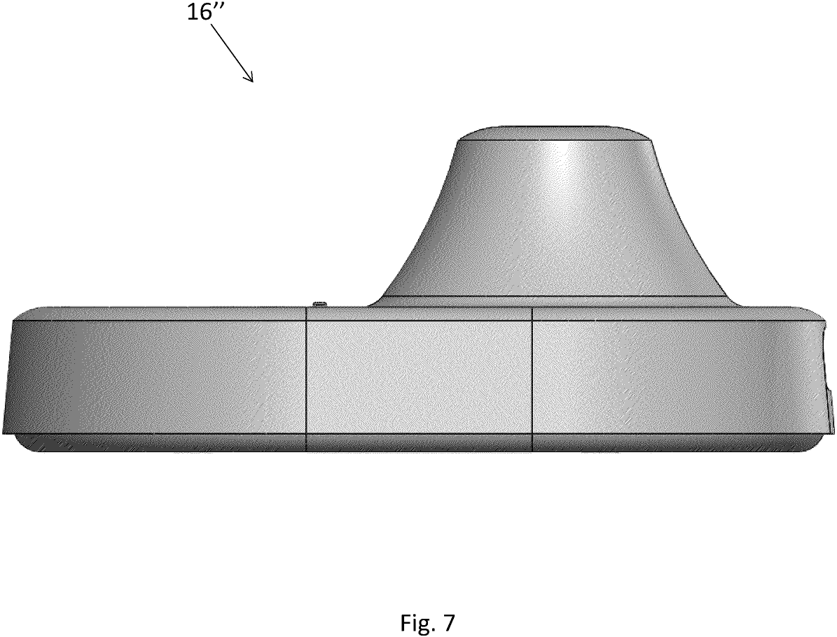

FIG. 7: is a side elevation view of the testing device of FIG. 6;

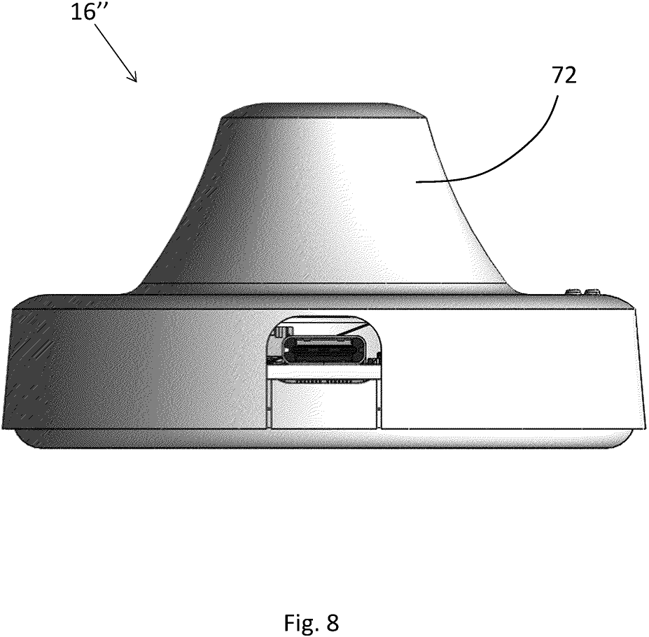

FIG. 8: is a front elevation view of the testing device of FIG. 6;

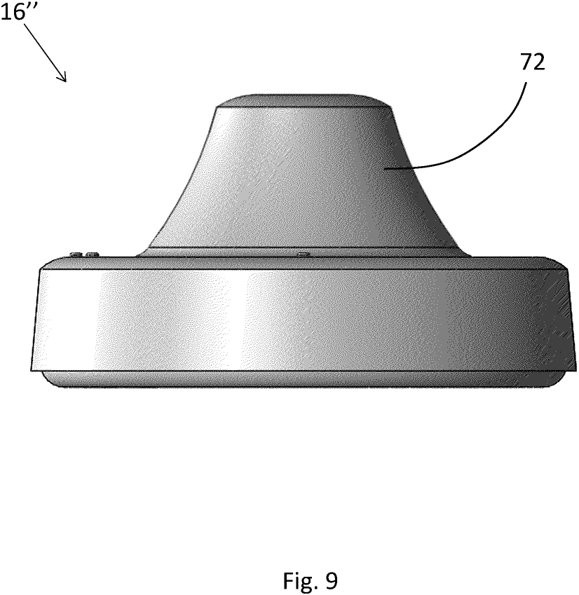

FIG. 9: is a back elevation view of the testing device of FIG. 6;

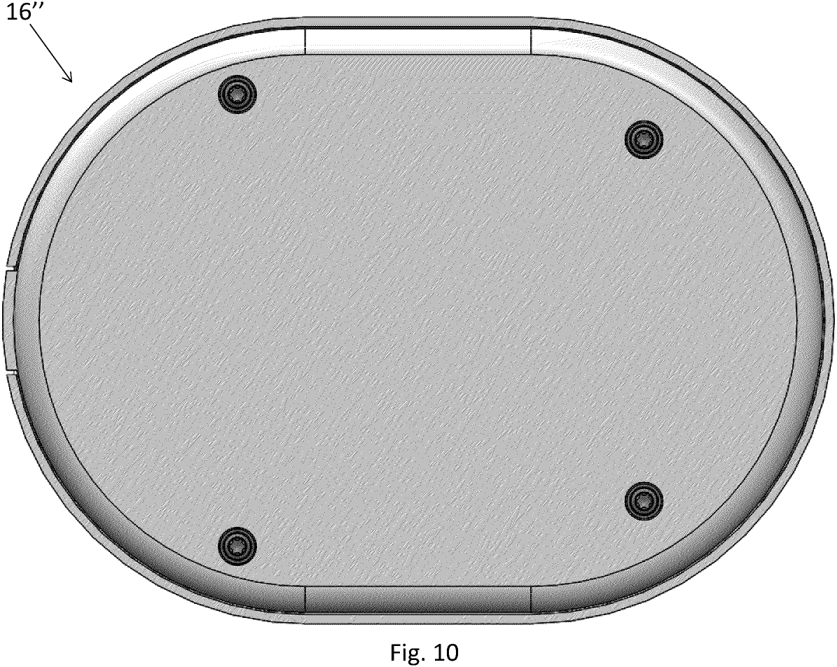

FIG. 10: is a bottom view of the testing device of FIG. 6;

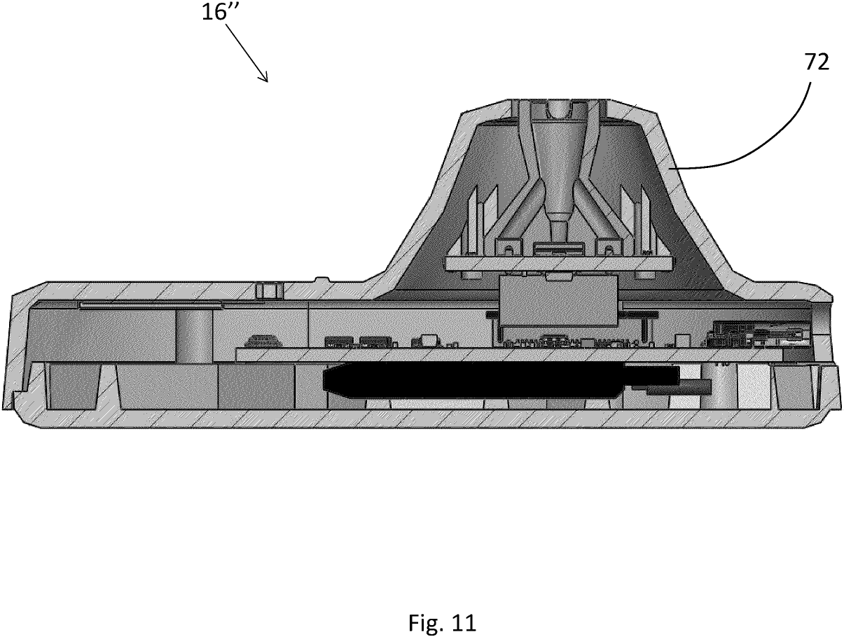

FIG. 11: is a cross-sectional view of the testing device of FIG. 6;

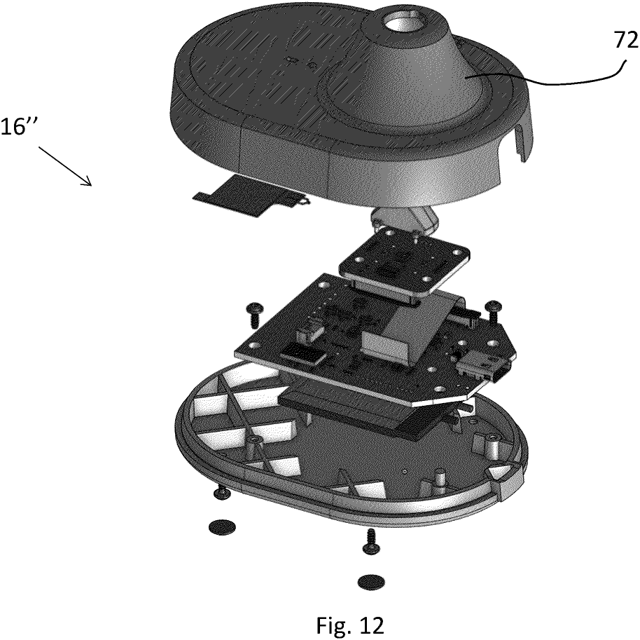

FIG. 12: is an exploded isometric view of the testing device of FIG. 6;

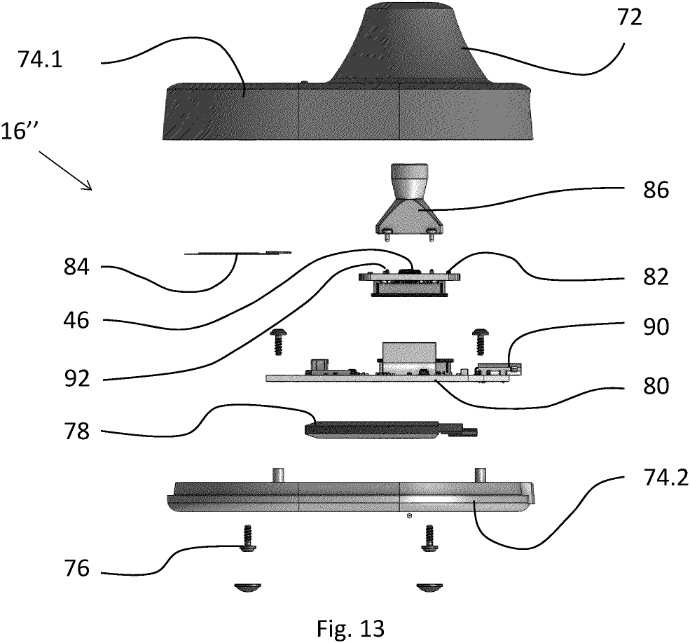

FIG. 13: is an exploded side view of the testing device of FIG. 6; and

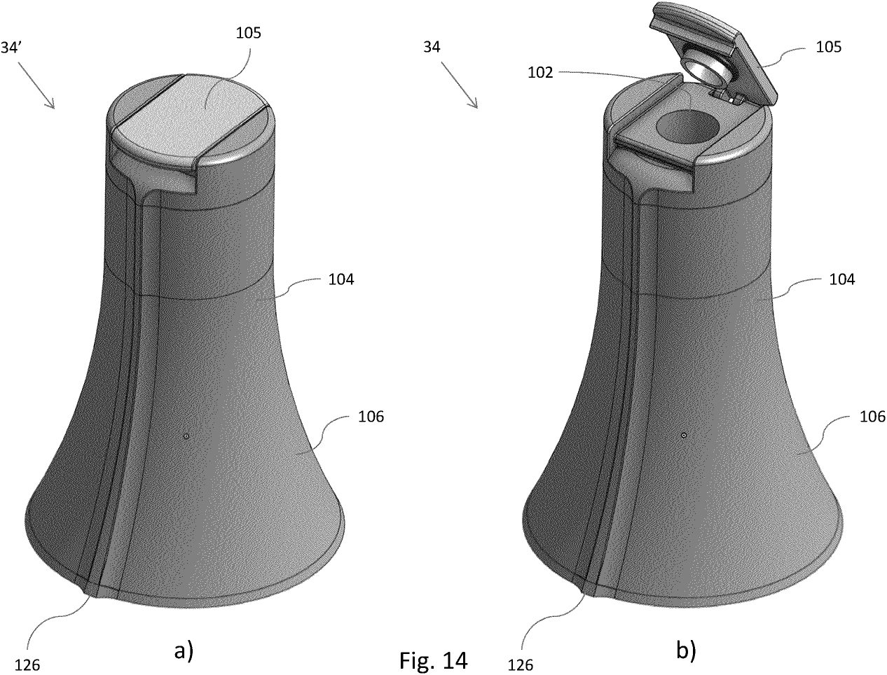

FIG. 14a: is an isometric view of a set of containers forming a fluid tight assembly to be used with the detection system of FIGS. 6 to 13;

FIG. 14b: is an isometric view of the fluid tight assembly from FIG. 14a with an open lid;

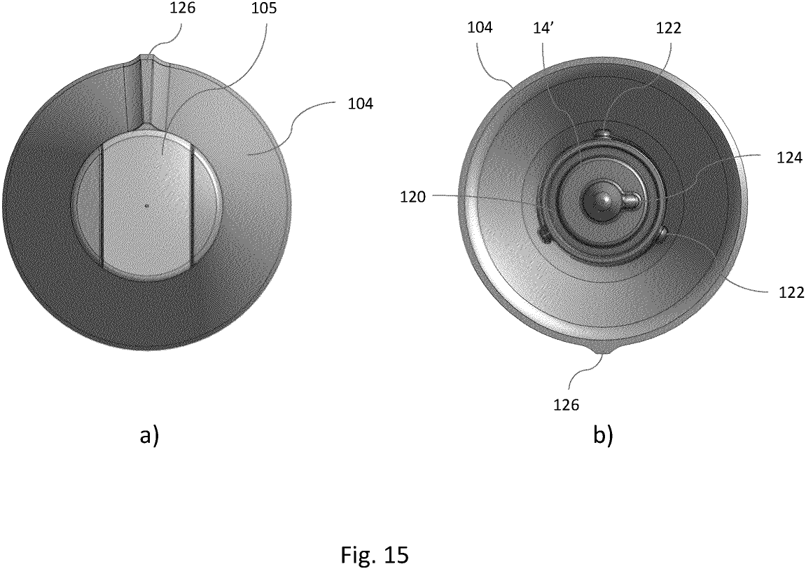

FIG. 15a: is a top view of the fluid tight assembly from FIG. 14;

FIG. 15b: is a bottom view of the fluid tight assembly from FIG. 14; and

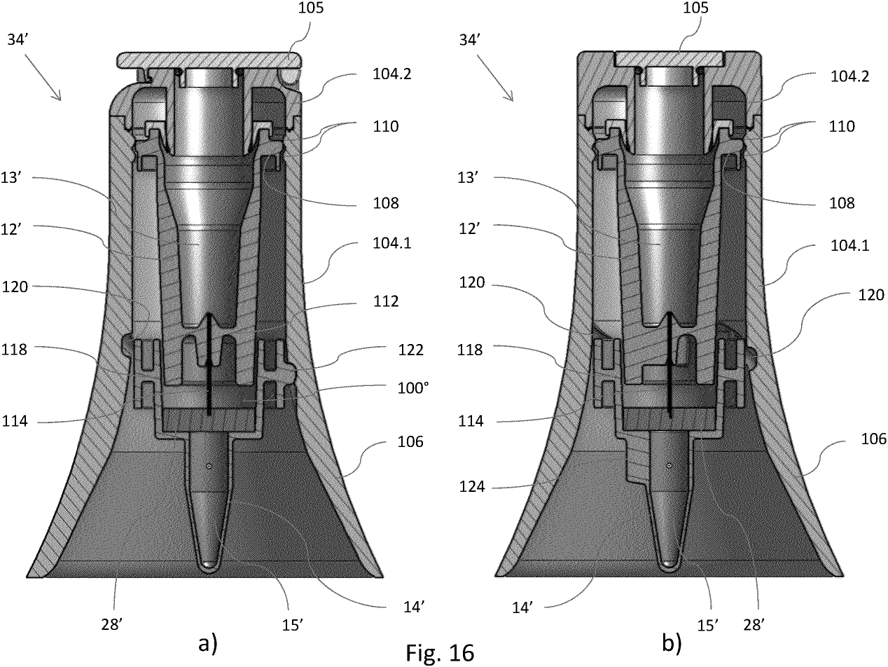

FIGS. 16a and b: are cross sectional views of the fluid tight assembly from FIGS. 14 and 15.

The system 10 for detecting a target analyte comprises a lysis chamber 12, a test chamber 14 (amplification chamber 14), a piston 16 and fluorescence detection device 18, see FIG. 1.

The amplification chamber 14, the lysis chamber 12 and the piston 18 form a tight assembly 34 that can be handled as a single, fluid tight unit; see FIG. 2.

The lysis chamber 12 contains a lysing fluid that causes lysing of cells or viruses in a sample to be tested. By way of lysing, nucleic acids such as DNA or RNA are released by way of breaking down the cells or viruses in the sample to be tested. Lysing can be achieved by a lysing fluid that comprises an acid such as hydrochloric acid or a weak alkali. The lysis chamber 12 has a lid 20 so lysis chamber 12 can be opened and a sample to be tested can be entered in the lysis chamber 12. The contents of the lysis chamber 12 is about 100 µl.

Lid of the lysis chamber preferably is a membrane 20 that can be pierced by a cotton swab with a sample.

The detection system 10 further comprises the amplification chamber 14. Amplification chamber 14 comprises a mixture of enzymes that are needed for a recombinase polymerase amplification (RPA). Preferably, the mixture is provided in the form of a dry pellet 26 that is contained in the amplification chamber 14.

The amplification chamber preferably is a cuvette that can be inserted in a receptacle 24 of the fluorescence detection device 16. The receptacle 22 is part of a detection chamber 26 of the fluorescence detection device 16.

The amplification chamber 14 is dimensioned to allow inserting the lysis chamber 12 into the amplification chamber in a tight and defined manner. An abutment 28 limits the depth of insertion. Once the lysis chamber 12 is fully inserted into the amplification chamber 14, the piston 18 can be used to transfer the fluid from the lysis chamber into the amplification chamber 14 and allowing the recombinase polymerase amplification to work in the closed amplification chamber 14. To allow the transfer of the contents of the lysis chamber 12 into the amplification chamber 14, a further thin lid 28 is arranged at the bottom of the lysis chamber 12. The thin lid 28 is dimensioned to break under the fluid pressure caused by the piston. Alternatively, the piston 18 and the thin lid 28 can be designed so that a tip (not shown) of the piston 18 can pierce the thin lid.

The lysis chamber 12, the amplification chamber 14 and the piston 18 are configured to engage in a fluid-tight manner when fully inserted. Such fluid tight engagement can be achieved by means of a snap fit connection wherein an annular protrusion 30 of one of the lysis chamber 12 and the amplification chamber 14 engages in an annular groove of the respective other chamber. Likewise, an annular protrusion 32 of one of the piston 18 and the lysis chamber 12 engages in an annular groove of the respective other part. Annular protrusions 30 and 32 act as sealings and may be integrally formed with the rest of the respective chamber or piston.

As an alternative to a snap fit connection, a screw lock connection similar to a Luer-lock can be provided for tightly connecting the lysis chamber 12 and the amplification chamber 14.

To allow insertion of the lysis chamber 12 into the amplification chamber 14 and of the piston 18 into the lysis chamber 12, venting means (not shown) are provided.

Once fully engaged, the amplification chamber 14, the lysis chamber 12 and the piston 18 form a tight assembly 34 that can be handled as a single, fluid tight unit.

Walls 36 of the amplification chamber 14 are transparent so as to allow light to enter the amplification chamber 14 and to exit the amplification chamber 14. The transparent walls 36 of the amplification chamber 14 make it possible to expose the content of amplification chamber 14 to exiting light that can cause a luminescence. In case, the content of the amplification chamber is luminescent luminescence of the sample in the amplification chamber 14 can be detected through the transparent walls 36 of the amplification chamber 14.

The pellet 26 that contains the mixture of enzymes forrecombinase polymerase amplification preferably comprises a recombinase, a single-stranded DNA-binding protein (SSB) and a strand-displacing polymerase, exonuclease III and in case RNA is to be detected, a reverse transcriptase.

In use, a sample to be tested first is filled into lysis chamber 12. After lysing the cells in the sample to be tested, the entire content of lysis chamber 12 is transferred into the amplification chamber 14 by means of the piston 18 so that a recombinase polymerase amplification can occur in the amplification chamber 14.

Once the recombinase polymerase amplification has occurred in the amplification chamber 14 — typically between 10 to 15 minutes after filling in the content of the lysis chamber 12 into the amplification chamber 14 — the amplification chamber 14 — or alternatively only is content — can be entered into the fluorescence detection device 18.

In the illustrated, preferred embodiment, the entire assembly 34 is inserted in the receptacle 22 of the fluorescence detection device 16. For handling of the assembly 34, a grip 38 is provided at a proximal end of the piston 18 see FIG. 3..

In order to prevent external light, for instance stray light, from entering into the receptacle once the assembly 34 is fully inserted in the receptacle 22, a collar 40 is provided that forms a lid for the receptacle 22.

The fluorescence detection device 16 comprises a detection chamber 42 that is configured to receive the amplification chamber 14 or the contents of the amplification chamber 14. In the illustrated, preferred embodiment, the receptacle 22 is part of the detection chamber 42. Within the detection chamber 42 or adjacent to the detection chamber 42 a light source 44 and an optical sensor 46 are arranged. The light source 44 is configured to illuminate the contents of the detection chamber 42 with a light that can cause luminescence in a sample to be tested during and after the sample has undergone recombinase polymerase amplification. The optical sensor 46 is arranged and configured to detect luminescence in the detection chamber 42 in case luminescence occurs.

To power up the light source 44 and the optical sensor 46, an energy supply 48 is provided, see FIG. 4. The energy supply 48 may comprise a battery, preferably a rechargeable battery. Alternatively or additionally, energy supply 48 may comprise a power interface for connecting the fluorescence detection device 16 to an external power supply. The power interface may be wire-bound or wireless. The energy supply 48 may also comprise solar cells for providing photovoltaic power supply.

The light source 44 and the optical sensor 46 are further connected to controller 50 that is configured to control the operation of the light source 44 and the optical sensor 46 and to further read out a sensor output signal provided by the optical sensor 46. Controller 50 can be a microcontroller or a state machine.

The controller 50 is operatively connected to a wireless data interface 52 that is configured to allow for a data communication between the microcontroller 50 and an external device such as a mobile phone or another device for data communication and data processing.

Preferably, the wireless data interface 52 is operatively connected to the controller 50, to the energy supply 48 and to a data memory 54 and is configured to provide for energy harvesting, data storage and data communication. In particular, the wireless interface 52 implements means for near field communication (NFC) and comprises a data bus such as a I2C data bus 56 for communication with the controller 50. The wireless data interface 52 preferably is configured to allow bidirectional data communication so as to transmit data generated by the fluorescence detection device 16 to an external device and to receive the control commands and/or software updates from an external device so that the fluorescence detection device 16 can be controlled and updated by way of an external device.

The wireless data interface 52 may also implement WIFI-communication as an alternative to near field communication. Another alternative is Bluetooth-communication.

Preferably, all electronic components that implement the wireless data interface 52, the controller 50 and the data memory 54 are arranged on a flat printed circuit board 60. The flat printed circuit board 60 can be part of a laminated card wherein the electronic components are arranged between two laminated cover sheets 62. Electric contacts 64 on the surface of one or both cover sheets 60 allow communication and energy transfer between the electronic components and further components such as the light source 44 or the optical sensor 46. Alternatively, in other embodiments, the printed circuit board can be flexible, semi-flexible or a rigid-flexible board. The printed circuit board may be a conventional circuit board or a circuit board manufactured by printing or other additive methods, or combinations thereof.

In addition to the electronic components, the printed circuit board 60 that can be laminated between two cover sheets 62 also comprises at least one antenna 66 for wireless data communication.

Optionally, heating means can be provided. The heating means may comprise an electric heating that is integrated into the fluorescence detection device 16 and that can heat the assembly 34 and its contents to thus start and/or promote the amplification process. Preferably, the fluorescence detection device 16 is configured to automatically start electric heating once the assembly 34 is inserted into the receptacle 22. The insertion of the assembly 34 can be detected by means of a dedicated sensor, for instance a switch, or by means of the optical sensor 46. In particular, the optical sensor 46 can sense that the assembly is inserted into the receptacle 22 because the collar 40 prevents external light from entering the detection chamber 42 once the assembly is inserted in the receptacle 22. Accordingly, insertion of the assembly 34 into the receptacle 22 causes a drop in light intensity sensed by the optical sensor 46. Such drop in light intensity can be detected and can cause a starting signal for the electric heating.

Alternatively or additionally, the heating means may be provided by chemicals in the amplification chamber 14 that undergo an exothermal reaction once a sample is filled in the amplification chamber 14. It is also possible that the lysis chamber 12 comprises a first component and the amplification chamber 14 comprises a second component wherein the two components can react exothermically. The exothermal reaction begins when the contents of the lysis chamber 12 is transferred into the amplification chamber 14.

Alternatively or additionally, the heating means may be provided by chemicals that undergo an exothermal reaction once a sample is introduced into the receptacle. These chemicals are arranged in proximity of the amplification chamber 14, in a separate sealed arrangement that contains the heating chemicals. Upon a trigger, the exothermic chemical reaction is started.

Another alternative that allows heating of the fluorescence detection device is proving a dark colour, for instance black, on the outside of the fluorescence detection device. Thus, the fluorescence detection device together with the amplification chamber can be solar heated. Preferably, temperature control means are provided that indicate potential overheating of the fluorescence detection device together with the amplification chamber. The temperature control means may comprise an ink or paint that changes its colour in case a predefined temperature is exceeded. Accordingly, an indicator that changes its colour at a certain temperature and that is applied to the outside of the fluorescence detection device may be a temperature control means.

The detection chamber 42 is arranged in a detection chamber housing 70 that has outer dimension smaller than 10 cm by 10 cm by 4 cm. Preferably the volume of the entire fluorescence detection device 16 is smaller than 200 cm³, and even more preferred smaller than 100 cm³. In a preferred embodiment, the longest outer dimension is a least twice as long as the shortest outer dimension.

The fluorescence detection device 16′ may provide a second receptacle 68 wherein the lysis chamber 12 can be placed prior to the connection with the amplification chamber 14; see FIG. 5. The second receptacle 68 can also host the amplification chamber 14 and the lysis chamber 12 prior to use.

The testing device 16′ can have an outer shape as shown in FIGS. 6 to 10. The receptacle 24′ is housed in a truncated cone shaped protrusion 72 on the top side of the testing device 16′ that also houses the detection chamber 42′ and thus is a detection chamber housing 70′.

The testing device 16′ has a two-part case 74 with a top part 74.1 and a bottom part 74.2 that are screwed together by four screws 76; see FIGS. 10 and 13.

Inside case 74, a rechargeable battery 78, a main printed circuit board (main PCB) 80, an auxiliary PCB 82 an NFC (near field communication) antenna 84 and a detection chamber enclosure 86 are arranged; see FIGS. 11 to 13.

The main PCB 80 and the auxiliary PCB 82 are electrically connected by means of a flexible flat cable 88; cf. FIG. 12. The main PCB 80 carries a USB-C port 90 that is used for charging the battery 78. The USB-C port is accessible from the front of the testing device 16′; cf. FIG. 8. The battery 78 is part the energy supply 48 of the testing device 16′.

The auxiliary PCB 82 carries two illuminating light emitting diodes (LED) 92 that are the light source 44 for the detection chamber 42′. The auxiliary PCB 82 further carries the optical sensor 46 that is arranged centrally between the LEDs 92. The detection chamber enclosure 86 covers the LEDs 92 and the optical sensor 46 and provides light paths 94 and 96 to the receptacle 24′ for the test camber 14′ (amplification chamber 14) when inserted into the testing device 16′.

The truncated cone shaped protrusion 72 matches the shape of the fluid tight assembly 34′ comprising the lysis chamber 12′ and test chamber 14′. The truncated cone shape prevents stray light from entering the receptacle 24′. Likewise, detection chamber enclosure 86 further prevents stray light from entering into the receptacle 24′.

The detection device 16″ is configured to match a fluid tight assembly 34′ as illustrated in FIGS. 14 to 16. The fluid tight assembly 34′ includes the lysis chamber 12′ and the test chamber 14′.

Lysis chamber 12′ and test chamber 14′ are hold in a housing 104 that is comprised of two parts, a lower housing part 104.1 and an upper housing part 104.2. The lower housing part 104.1 has a flared side wall 106 that widens towards the bottom of housing 104. The flared side wall 106 is configured to fit to a truncated cone shaped protrusion 72 of the detection device 16″ (see FIGS. 8, 9, 11, 12 and 13).

The top of housing 104 is closed by a cap 105 that can swivel into an open position (see FIG. 14b) for selectively opening a central opening 102 for inserting a swab into the lysis chamber 13′ of lysis container 12′.

In the upper part of housing 104 the lysis container 12′ is held by means of a releasable snap fit connection achieved by a outwardly extending circumferential collar 108 around an upper section of the lysis container outer wall and two inwardly extending circumferential rips 110 on the inner wall of an upper section of housing 104. The outwardly extending circumferential collar 108 of the lysis container 12′ is held in a grove between the two inwardly extending circumferential rips 110 on the inner wall of housing 104. The snap fit connection between the lysis chamber 12′ and the housing 104 can be released by a force acting on the lysis chamber 12′ in an axial direction that exceeds a predetermined threshold as provided by the design of the snap fit connection as provided inter alia by the matching shapes and the elastic properties of the materials and shapes.

In the bottom 112 of the lysis container 12′ a hollow needle 114 is arranged. The needle 114 preferably is made from stainless steel.

The lower part of the lysis container 12′ including the lysis container bottom 112 is arranged to extend into a lysis container receptacle 116 that is formed by a test container extension 118 extending upwardly from an upper part of the test container 14*. The outer diameter of the lower part of the lysis container 12′ corresponds to the inner diameter of the lysis container receptacle 116.

A rotation of the housing 104 relative to the test container 14′ enclosing the test chamber 15′ causes a relative axial movement of the test container 14′. This is achieved by at least one helical grove 120 that is formed in the inner side of the wall of the housing 104. In the illustrated embodiment, three helical groves 120 are provided. A radial protrusion 122 on the test container extension 118 radially extends into the grove 120. Thus, the grove 120 acts as a helical slotted link guide for the radial protrusion 122 on the test container extension 118. The helical grove 120 causes an axial movement of the test container 14′ when the housing 104 is rotated while the test container 14′ does not rotate.

In order to prevent a rotation of the test container 14′ when the housing 104 is rotated, a short longitudinal rip 124 extending in the radial direction from a wall of the test container 14′ is provided. The rip 124 engages with a notch 98 of the receptacle 22′ of the detection device 16′ when the test container 14′ of fluid tight assembly 34′ is inserted on the receptacle 22′ of the detection device 16′. The notch 98 at the circumferential inner edge 100 of the receptacle 24′ is configured to interact with a lateral protrusion on the test chamber 14′ to prevent the test chamber 14′ from rotating when an outer flared side wall 106 (see FIGS. 14 and 16) of the fluid tight assembly 34′ is rotated by hand in order to move the test chamber 14′ towards the lysis chamber 12′.

Rotating of the housing 104 relative to the test container 14′ causes an axial movement of the test container 14′ towards the lysis container 12′. This in turn causes the needle 114 to pierce the elastomeric septum 28′ (i.e. the lid) of the test container 14′. Once the needle 114 at the bottom 112 of lysis container 12′ pierces the elastomeric septum 28′ (separating wall acting as a lid for the test container that can be pierced by a needle) that initially closed the second chamber in the test container 14′, fluid can be transferred from the lysis container 12′ into the test container 14′.

The axial movement of the test container 14′ towards the lysis container 12′ further causes a compression of the lysis container 12′ and thus a transfer of fluid from the lysis chamber 13′ into the test chamber 15′. Further rotating of housing 104 causes further compression of the lysis container 12′ until the axial forces on the lysis container 12′ causing the compression of the lysis container 12′ exceed the force needed to release the lysis container 12′ from being held by the outwardly extending circumferential collar 108 of the lysis container 12′ in the grove between the two inwardly extending circumferential rips 110 on the inner wall of housing 104. Once released, the lysis container 12′ can freely move upwards (i.e. in the direction towards the central opening 102 at the top of housing 104) and the lysis container 12′ is not further compressed. Thus, further transfer of fluid from the lysis chamber 13′ into the test chamber 15′ is stopped. Compression of the lysis container 12′ is thus limited by the force needed for pushing the outwardly extending circumferential collar 108 of the lysis container 12′ out of the grove between the two inwardly extending circumferential rips 110 on the inner wall of housing 104.

For using the fluid tight assembly 34′ in combination with the detection device 16′, first a swap with a sample to be tested is inserted in the lysis camber 13′ via the central opening 102. Next, cap 105 is closed and lysis can occur in the lysis chamber 13′. Once lysis has occurred, the fluid tight assembly 34′ can be engaged with the detection device 16′ by inserting the test container 14′ into the receptacle 22′ of the detection device 16′. This is facilitated by the flared side wall 106 of the lower section of housing 104.

To further facilitate engaging the fluid tight assembly 34′ with the detection device 16′ in the right orientation that allows the short longitudinal rip 124 of the test container 14′ to engage with the notch 98 of the receptacle 22′ of the detection device 16′, a palpable raised rip 126 is provided on the outer surface of the housing 104. The palpable raised rip 126 runs along a longitudinal direction of the housing 104.

A palpable protrusion 128 on the detection device 16′ next to the truncated cone shaped protrusion 72 of the detection device 16′ is a further palpable feature that helps orienting the palpable raised rip 126 and thus the fluid tight assembly 34′ correctly.

Once engaged with the detection device 16′, the housing 104 of the fluid tight assembly 34′ can be rotated to cause the relative axial movement of the test container 14′ relative to the lysis container 12′ and the housing 104. Since immediately after engaging the fluid tight assembly 34′ with the testing device 16′′ the test container 14′ already is fully inserted into the receptacle 22′ and thus the detection chamber 42′ of the detection device 16′, the housing 104 is axially spaced from the detection device 16′. By rotating the housing 104 relative to the detection device 16′ and thus the test container 14′, the axial distance between the housing 104 and the detection device 16′ is minimized until the housing 104 touches the detection device 16′ and the needle 114 has pierced the septum 28′ of the test container 14′. As seen from outside, while rotating the housing 104 clockwise, the housing 104 moves downwardly thus approaching the detection device 16′.

Once the housing 104 is rotated in its final position, a further effect of the truncated cone shaped protrusion 72 of the detection device 16′ in combination with the flared side wall 106 of the housing 104 is an improved protection from straylight entering the detection chamber 42′ of the detection device 16′.

The detection chamber 42′ of the fluorescence detection device 16′ is configured to receive the test container 14′. The receptacle 22′ is part of the detection chamber 42′.

Providing a main printed circuit board (main PCB) 80 and a separate auxiliary PCB 82 that carries the illuminating light emitting diodes (LED) 92 and the optical sensor 46 has the advantage that forces acting on the detection chamber enclosure 86 when the test container 14° is inserted in the detection chamber 42 cannot be transferred to the main PCB 80 since the main PCB 80 and the auxiliary PCB 82 are connected by means of the flexible flat cable 88.

1. A system for detecting target DNA in a biological sample, said system comprising a lysis container with a lysis chamber, a test container with an amplification chamber and fluorescence detection device, wherein:
the lysis chamber contains a lysing fluid,
the amplification chamber contains a mixture that comprises a recombinase, a single-stranded DNA-binding protein, strand-displacing polymerase and exonuclease, and
the fluorescence detection device comprises:
a detection chamber configured to receive the amplification chamber or the contents of the amplification chamber,
a light source,
an optical sensor,
energy supply means,
a wireless data interface, and
a controller.

2. A set of at least two distinct chambers that can be combined to form a single, fluid tight assembly, wherein a first chamber comprises a first set of chemicals and/or agents, said first chamber being closed prior to use, and wherein a second chamber comprises a second set of chemicals and/or agents that are at least in part distinct from the chemicals and/or agents of the first set, and wherein the first chamber comprises a lid, that can be opened when the first chamber and the second chamber are combined to form a single, fluid tight assembly, in order to allow the contents of the first chamber to enter the second chamber.

3. The set of chambers according to claim 2, wherein each chamber is an integral unibody.

4. The set of chambers according to claim 3, wherein the chambers can be connected by means of a fluid tight snap fit connection.

5. The set of chambers according to claim 2, wherein the assembly of the first chamber and the second chamber is an integral unibody.

6. The set of chambers according to claim 2, wherein the second chamber has transparent walls.

7. A fluorescence detection device, comprising:
a detection chamber configure to receive an amplification chamber or the contents of the amplification chamber,
a light source configured to excite luminescence and in particular fluorescence,
an optical sensor configured to capture luminescence and on particular fluorescence,
energy supply means,
a wireless data interface, and
a controller.

8. The fluorescence detection device according to claim 7, further comprising a receptacle that is formed to receive an amplification chamber.

9. The fluorescence detection device according to claim 7, further comprising heating means.

10. The fluorescence detection device according to claim 7, wherein the optical sensor and the light source are arranged on a circuit board that is separate from a circuit board comprising the controller.

11. The fluorescence detection device according to claim 7, wherein the detection device comprises a receptacle for receiving a test container said receptacle comprising an abutment that prevent a relative rotation between the detection device and the test container when the test container is inserted into the receptacle.
